(19) United States
(12) Patent Application Publication
(10) Pub. No.: US 2013/0204270 A1
(43) Pub. Date: Aug. 8, 2013

(54) INSERTION ASSEMBLY FOR AN ELECTRICAL STIMULATION SYSTEM AND RELATED METHODS OF USE

(71) Applicant: BOSTON SCIENTIFIC NEUROMODULATION CORPORATION, Valencia, CA (US)

(72) Inventors: Joshua Dale Howard, North Hollywood, CA (US); Roger Chen, Los Angeles, CA (US); Carolyn Madeleine Nohejl, Los Angeles, CA (US); Aditya Vasudeo Pandit, Costa Mesa, CA (US); Anne Margaret Pianca, Santa Monica, CA (US); John Michael Barker, Ventura, CA (US)

(73) Assignee: BOSTON SCIENTIFIC NEUROMODULATION CORPORATION, Valencia, CA (US)

(21) Appl. No.: 13/760,743

(22) Filed: Feb. 6, 2013

Related U.S. Application Data
(60) Provisional application No. 61/595,568, filed on Feb. 6, 2012.

(51) Int. Cl. A61N 1/05

(52) U.S. Cl. CPC A61N 1/05
USPC 606/129; 607/116

(57) ABSTRACT

An insertion assembly for assisting implantation of at least one lead into a patient includes an insertion needle and a sheath. The sheath removably couples with the insertion needle while the insertion needle is being advanced into the patient. When the sheath is coupled to the insertion needle, the sheath is disposed over at least a portion of an outer surface of the insertion needle. The sheath is radially expandable from a non-expanded state to an expanded state that is rigid enough to retract surrounding patient tissue when inserted into the patient. When the sheath is in an expanded state, first and second diameters of the sheath at opposing ends of the sheath are each large enough to concurrently receive at least one of a paddle lead or at least two percutaneous leads.

### CROSS-REFERENCE TO RELATED APPLICATIONS

This application claims the benefit under 35 U.S.C. §119(e) of U.S. Provisional Patent Application Ser. No. 61/595,568 filed on Feb. 6, 2012, which is incorporated herein by reference.

### FIELD OF THE INVENTION

The present invention is directed to the area of implantable electrical stimulation systems and methods of making and using the systems. The present invention is also directed to implantable electrical stimulation leads and insertion assemblies with expandable members for assisting implantation of the leads in patients.

### BACKGROUND

Implantable electrical stimulation systems have proven therapeutic in a variety of diseases and disorders. For example, spinal cord stimulation systems may be implanted in the spinal cord to treat chronic pain syndromes and in the brain to treat refractory chronic pain syndromes, movement disorders, and epilepsy. Peripheral nerve stimulation systems may be used to treat chronic pain syndrome and incontinence. In some cases, paralyzed extremities in spinal cord injury patients may be treated using functional electrical stimulation. Moreover, electrical stimulation systems can be implanted subcutaneously to stimulate subcutaneous tissue including subcutaneous nerves such as the occipital nerve.

In general, a stimulator includes a control module (with a pulse generator), one or more leads, a paddle body connected to the distal end of the lead(s), and an array of stimulator electrodes mounted on the paddle body. The stimulator electrodes are placed in contact with or near the nerves, muscles, or other tissue to be stimulated. The pulse generator in the control module generates electrical pulses that are delivered through the electrodes to body tissue.

### BRIEF SUMMARY

In one embodiment, an insertion assembly for assisting implantation of at least one lead into a patient includes an insertion needle and a sheath. The insertion needle has an outer surface and is configured and arranged for advancing into the patient. The sheath has a proximal end with a first diameter, a distal end with a second diameter, and a lumen extending between the proximal and distal ends. The sheath is radially expandable from a non-expanded state to an expanded state that is rigid enough to retract surrounding patient tissue when the sheath is inserted into the patient during implantation. The sheath is configured and arranged for removably coupling with the insertion needle while the insertion needle is being advanced into the patient. When the sheath is coupled to the insertion needle, the sheath is disposed over at least a portion of the outer surface of the insertion needle. When the sheath is in an expanded state, the first and second diameters are each large enough to concurrently receive at least one of a paddle lead or at least two percutaneous leads.

In another embodiment, an insertion assembly for assisting implantation of at least one lead into a patient includes a guidewire, an insertion needle, a tissue-separating member, and a sheath. The guidewire is configured and arranged for insertion into the patient. The insertion needle is configured and arranged for advancing into a patient via the guidewire. The insertion needle has a proximal end and a distal end, and defines a lumen extending between the proximal and distal ends. The lumen is configured and arranged to receive the guidewire. The tissue-separating member has a proximal end, a distal end, and an outer surface. The tissue-separating member defines a lumen extending between the proximal and distal ends. The lumen is configured and arranged for receiving the guidewire. The sheath has a proximal end with a first diameter, a distal end with a second diameter, and a longitudinal length. The sheath defines a lumen extending between the proximal and distal ends. The sheath is radially expandable from a non-expanded state to an expanded state that is rigid enough to retract surrounding patient tissue when the sheath is inserted into the patient during implantation. The sheath is configured and arranged for removably coupling with the tissue-separating member while the tissue-separating member is being advanced into the patient. When the sheath is coupled to the tissue-separating member, the sheath is disposed over at least a portion of the outer surface of the tissue-separating member. When the sheath is in an expanded state, the first and second diameters are each large enough to concurrently receive at least one of a paddle lead or at least two percutaneous leads.

### BRIEF DESCRIPTION OF THE DRAWINGS

Non-limiting and non-exhaustive embodiments of the present disclosure are described with reference to the following drawings. In the drawings, like reference numerals refer to like parts throughout the various figures unless otherwise specified.

For a better understanding of the present disclosure, reference will be made to the following detailed description, which is to be ready in association with the accompanying drawings, wherein:

FIG. 1 is a schematic perspective view of another embodiment of the sheath of FIG. 10, according to the invention;

FIG. 2 is a schematic perspective view of yet another embodiment of the sheath of FIG. 10 with multiple handles and a pull string, according to the invention;

FIG. 3 is a schematic side view of one embodiment of a proximal portion of the sheath of FIG. 10 and a distal portion of a tissue-separating member suitable for insertion into the sheath, according to the invention;

FIG. 4 is a schematic side view of one embodiment of a guiding member coupled to the sheath of FIG. 10, according to the invention;

FIG. 5 is a schematic side view of one embodiment of the tissue-separating member of FIG. 3 partially disposed in the sheath of FIG. 3, according to the invention;

FIG. 6A is a schematic perspective view of another embodiment of the sheath of FIG. 10 having an expandable flange coupled to a portion of the sheath and an adjustable locking mechanism for retaining the flange at a desired level of expansion, the locking mechanism in an unlocked position, according to the invention;

FIG. 6B is a schematic perspective view of a lead with a junction partially disposed in the sheath of FIG. 6A, the locking mechanism retaining the flange of the sheath at an expanded state for receiving the junction of the lead, according to the invention;

FIG. 7 is a schematic side view of one embodiment of the sheath of FIG. 10 having a distally-tapering body, according to the invention;

FIG. 8A is a schematic view of one embodiment of an insertion needle inserted into an epidural space of a patient, according to the invention;

FIG. 8B is a schematic view of one embodiment of a guidewire inserted through the insertion needle of FIG. 8A, according to the invention;

FIG. 8C is a schematic view of one embodiment of the insertion needle of FIG. 8A being removed from a proximal end of the guidewire of FIG. 8B, according to the invention;

FIG. 8D is a schematic view of one embodiment of an insertion assembly being disposed over the guidewire of FIG. 8B, the insertion assembly including the sheath of FIG. 7 disposed over a dilator, according to the invention;

FIG. 8E is a schematic view of one embodiment of the insertion assembly of FIG. 8D guided along the guidewire of FIG. 8B and disposed at an epidural space of a patient, according to the invention;

FIG. 8F is a schematic view of one embodiment of the dilator of FIG. 8D partially inserted into the sheath of FIG. 8D, according to the invention;

FIG. 8G is a schematic view of one embodiment of the dilator of FIG. 8D fully inserted into the sheath of FIG. 8D, thereby causing the sheath to expand radially, according to the invention;

FIG. 8H is a schematic view of one embodiment of a lead extending through the sheath of FIG. 8D with a distal end of the lead disposed in the epidural space of a patient, according to the invention;

FIG. 8I is a schematic view of one embodiment of the sheath of FIG. 8D being removed from a proximal end of the lead of FIG. 8H, according to the invention;

FIG. 9 is a schematic perspective view of a stimulation system suitable for insertion into a patient, according to the invention; and

FIG. 10 is a schematic side view of one embodiment of an insertion assembly suitable for use facilitating implantation of the stimulation system of FIG. 9 into a patient, the insertion assembly including an expandable sheath disposed over a portion of an insertion needle, according to the invention.

Corresponding reference characters indicate corresponding components throughout the several views of the drawings.

### DETAILED DESCRIPTION

Embodiments of the present disclosure relate to implantable electrical stimulation systems and related methods of use. The disclosure is also directed to systems and methods for concurrently implanting one or more leads into a single subcutaneous passage.

Suitable implantable electrical stimulation systems include, but are not limited to, a lead with one or more electrodes disposed on a distal end of the lead and one or more terminals disposed on one or more proximal ends of the lead. Leads include, for example, percutaneous leads, paddle leads, and cuff leads. Examples of electrical stimulation systems with leads are present in, for example, U.S. Pat. Nos. 6,181,969; 6,516,227; 6,609,029; 6,609,032; 6,741,892; 7,244,150; 7,672,734; 7,761,165; 7,949,395; 7,974,706; and 8,175,710; and U.S. Patent Application Publications Nos. 2005/0165465 and 2007/0150036, all of which are incorporated by reference.

In the following sections, embodiments of the present disclosure will be described with reference to spinal cord stimulation. It will be understood that this choice is merely exemplary and that the device may be utilized in any other organ, such as the brain, urinary system, or any other organ or tissue that can be stimulated.

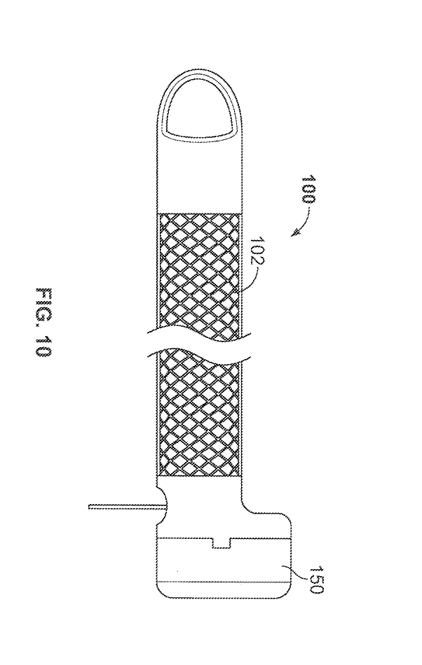

FIG. 9 illustrates one embodiment of a stimulation system 900. Stimulation system 900 includes a control module 902, such as a stimulator or pulse generator, a paddle body 904, and at least one lead body 906 coupling the control module 902 to the paddle body 904. One or more components of stimulation system 900 are typically implanted into a patient's body. The stimulation system can be used for a variety of applications including, but not limited to, brain stimulation, neural stimulation, spinal cord stimulation, muscle stimulation, and the like.

Paddle body 904 along with lead body 906 forms a paddle lead 908. The paddle body 904 and lead body 906 may be a unitary structure or these two components be formed as separate structures that are permanently or detachably coupled. Lead body 906 may be, for example, a round or tubular lead body having a proximal end 910 and a distal end 912 with at least one electrical conductor (not shown) extending between the proximal end 910 and distal end 912. The proximal end 910 of the lead body includes an array of terminal contacts (not shown) which are coupled to the electrical conductors and, through the conductors, to the electrodes described below.

Paddle body 904 is, in at least some embodiments, a generally flat body provided on the distal end of lead 906. In at least some embodiments, the paddle body has two major surfaces defining, respectively, a first face 914 and a second face 916. Typically, the first and second faces are on opposite sides of the paddle body. The first face 914 includes an array of electrodes 918, which are in electrical communication with the electrical conductor. In some embodiments, the second face 916 may also include one or more electrodes.

Electrodes 918 can be formed using any conductive, biocompatible material. Examples of suitable materials include metals, alloys, conductive polymers, conductive carbon, or combinations thereof. The number of electrodes in the array may vary. For example, there can be one, two, four, eight, ten, twelve, sixteen, or more electrodes. Further, the electrodes may be disposed on one or both faces of the paddle body. In at least some embodiments, the electrodes are arranged in an array of one or more columns and one or more rows. Alternatively, electrodes may be arranged in any other regular or irregular arrangement on the paddle body.

Electrical current is provided through one or more of the electrodes 918 to stimulate nerve fibers, muscle fibers, or other body tissues near the stimulation system. In one embodiment, a processor, within the control module 902, is included to control the timing and electrical characteristics of the stimulation system. For example, the processor can, if desired, control one or more of the timing, frequency, strength, duration, and waveform of the pulses. In addition, the processor can select which electrodes can be used to provide stimulation.

It will be understood that the stimulation system 900 can include more, fewer, or different components and can have a variety of different configurations known to those skilled in the art. For example, instead of paddle body 904, the electrodes 918 can be disposed in an array at or near the distal end of lead body 906 forming a percutaneous lead. Percutaneous lead may be isodiametric along the length of the lead. In addition, one or more lead extensions (not shown) can be disposed between the lead body 906 and control module 902 to extend the distance between paddle body 904 and control module 902. Further, paddle body 904 may be elliptical or tubular in shape.

In the case of implanting leads for spinal cord stimulation, the lead is advanced into the patient until the distal end of the lead is located within the epidural space of the patient and the electrodes of the lead are in operational communication with the desired spinal cord segment(s). In the case of isodiametric percutaneous leads, implantation may involve using an introducer needle, such as an epidural needle, to introduce the lead into the patient.

In the case of non-isodiametric leads (e.g., paddle leads and percutaneous leads having one or more larger-diameter sections, such as one or more junctions), implantation may involve performing a laminectomy. A laminectomy involves removal of the laminar vertebral tissue to allow access to the dura layer, followed by implementation of the lead. A laminectomy is an invasive procedure and may involve a lengthy recovery period for a patient. Accordingly, it may be advantageous to be able to implant non-isodiametric leads using an introducer needle to reduce patient trauma associated with implantation. Additionally, such implantation techniques may reduce procedure times and reduce health-care costs associated with lead implantations.

In some cases, multiple leads may be implanted into a patient. Unfortunately, conventional introducer needles do not create a passage through patient tissue that is large enough to accommodate concurrent implantation of multiple leads. Accordingly, it may be advantageous to be able to concurrently implant multiple leads using a single introducer needle to reduce patient trauma associated with implantation.

As herein described, an insertion assembly can be used to concurrently implant one or more non-isodiametric leads (e.g., paddle leads and percutaneous leads having one or more larger-diameter sections, such as a junction), one or more percutaneous leads, or some combination thereof into an epidural space of a patient. The insertion assembly includes a sheath that is inserted into the patient and expanded to enlarge a passage (e.g., a path, tunnel, or the like) to a target stimulation location (e.g., one or more desired spinal cord segments) within the epidural space of the patient. In at least some embodiments, the one or more leads may be implanted without having to cut away any ligaments, bones, or muscles. Once the sheath is expanded, one or more leads may be extended into the sheath and implanted at the target stimulation location.

The insertion assembly includes an insertion needle for facilitating advancement of the sheath into the patient. In at least some embodiments, the insertion assembly further includes a guidewire. Optionally, the insertion assembly may include one or more tissue-separating members (e.g., a passing elevator, a dilator, or the like or combinations thereof). In at least some embodiments, the sheath is disposed over at least a portion of the insertion needle. In other embodiments, the sheath is disposed over at least a portion of the tissue-separating member. Optionally, the insertion assembly may include a guiding member for facilitating guidance of the one or more leads to the target stimulation location.

FIG. 10 illustrates a schematic side view of one embodiment of an insertion assembly 100. The insertion assembly 100 includes an expandable sheath 102 coupled to the insertion needle 150 such that the sheath 102 is disposed over a portion of an outer surface of the insertion needle 150. The insertion needle 150 may include any needle suitable for inserting into a patient to form a passage in patient tissue of suitable size for insertion of an electrical stimulation lead. In at least some embodiments, the insertion needle 150 is a conventional epidural needle, such as a standard 14-gauge, 16-gauge, 18-gauge, 20-gauge, or smaller epidural needle. The sheath 102 can be formed from any suitable biocompatible materials including, for example, stainless steel, shape memory materials, polyurethane, or the like or combinations thereof.

The sheath 102 is configured and arranged to separate from the insertion needle 150 and expand. In at least some embodiments, the sheath 102 may be expanded after insertion of the insertion assembly 100 into a subcutaneous passage within a patient. In which case, the expansion of the sheath 102 enlarges the passage. After the insertion needle 150 is removed, the expanded passage provides space for one or more leads to be guided along the sheath to a target stimulation location.

The sheath 102 is configured and arranged to expand from a first state to a second state. In at least some embodiments, the sheath 102 is disposed in the first state when the sheath 102 is coupled to the insertion needle 150 and disposed in the second state when the one or more leads are disposed in the sheath 102. In at least some embodiments, when the sheath 102 is coupled to the insertion needle 150, the sheath 102 is in a relaxed state. In other embodiments, when the sheath 102 is coupled to the insertion needle 150, the sheath 102 is compressed. In at least some embodiments, the sheath 102 transitions between the first state and the second state upon application of a force. In other embodiments, the sheath 102 transitions between the first state and the second state upon removal of a force.

In at least some embodiments, a procedure for implanting the one or more leads into the epidural space of a patient includes advancing the insertion assembly 100 into a patient until a distal end of the insertion needle 150 is in positioned within the epidural space. In at least some embodiments, a loss of resistance test (or other suitable test) is performed to ensure placement of the distal end of the insertion needle 150 within the epidural space. In at least some embodiments, the positioning of the insertion needle 150 may be adjusted such that the distal end of the sheath 102 is also positioned within the epidural space.

The sheath 102 is detached from the insertion needle 150 and expanded radially. The sheath 102 can be detached from the insertion needle 150 and expanded radially in any suitable way including, for example, removing a force keeping the sheath 102 compressed against the insertion needle 150 (e.g., pulling a pull string to remove a cover, or one or more bands retaining the sheath 102 in a compressed state, a pull-away member, one or more clamps, or the like), or applying a force to expand the sheath 102 (e.g., inserting a tissue-separating member into the sheath, pulling apart pull-tabs coupled to the sheath, or the like), or combinations thereof. Note that when the sheath 102 is in a relaxed state during insertion, the sheath 102 may or may not need to be detached from the insertion needle 150 prior to expansion. Once the distal end of the sheath 102 is positioned at the epidural space and the sheath 102 is expanded radially, the insertion needle 150 may be removed from the patient.

One or more leads are inserted into the expanded sheath 102 and advanced along the lumen of the sheath 102 until the electrodes of the one or more leads are positioned in the epidural space. In at least some embodiments, one or more tissue-separating members (e.g., a passing elevator, a dilator, one or more balloons, or the like) may be used to facilitate expansion of the passage formed by the insertion needle 150 either before, or during, insertion of the one or more leads into the patient. The tissue-separating member may be inserted into the sheath 102 in any suitable manner including, for example, by itself, with the one or more leads, with a stiffening rod, or the like or combinations thereof.

When the distal ends of the one or more leads are positioned in the epidural space, the sheath 102 may be removed from the patient. Optionally, the distal end of the one or more leads may be further guided within the epidural space in order to position the distal end of the one or more leads in proximity to the target stimulation location(s). This may happen before the sheath is removed, after the sheath is removed, or both. In at least some embodiments one or more stiffening members (e.g., a lead blank, a stylet, or the like) may be used to facilitate guidance of the one or more leads. In at least some embodiments, a guiding member (402 in FIG. 4) may be advanced from a distal end of the sheath 102 to facilitate guidance of the sheath 102 and one or more leads to the target stimulation location(s) once the insertion needle 150 is removed.

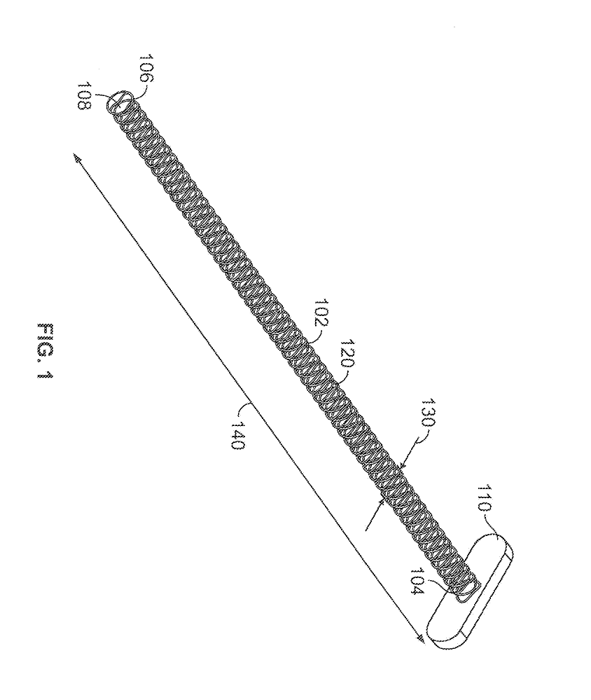

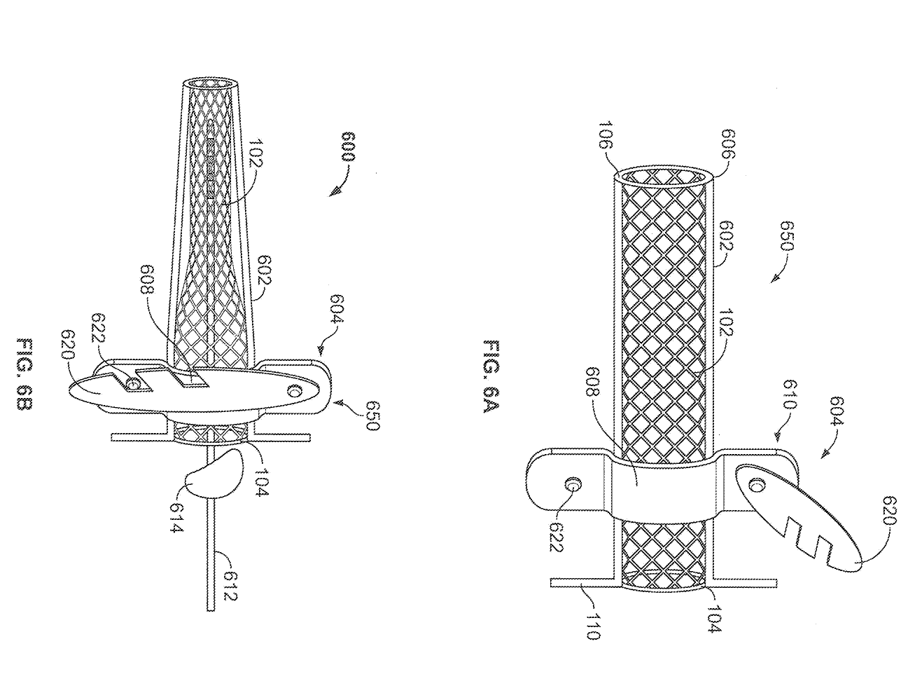

FIG. 1 illustrates a schematic perspective view of one embodiment of the sheath 102. The sheath 102 has a proximal end 104, a distal end 106, and a lumen 108 extending from the proximal end 104 to the distal end 106. The sheath 102 has a cross-sectional diameter 130 and a longitudinal length 140. In at least some embodiments, the sheath 102 is isodiametric. In some other embodiments (see e.g., FIG. 7), the sheath 102 tapers from the proximal end 104 to the distal end 106. The lumen 108 can have any suitable cross-sectional shape including, for example, round, oval, triangular, rectangular, or the like.

The sheath 102 is configured and arranged to expand radially. The sheath 102 may expand by any suitable amount. In at least some embodiments, when the sheath 102 expands, the diameter 130 of the sheath 102 increases by at least 10%, 20%, 30%, 40%, 50%, 60%, 70%, 80%, 90%, 100%, 120%, 140%, 160%, 180%, 200% or more from the diameter of the sheath 102 when the sheath 102 is disposed in a relaxed, or compressed, state.

Optionally, a handle 110 is coupled to the proximal end 104 of the sheath 102. The handle 110 may be used for facilitating the guiding of the sheath 102 once the sheath 102 is separated from the insertion needle 150. In at least some embodiments, the handle 110 is adapted to facilitate grasping of the proximal end 104 of the sheath 102 by a medical practitioner. The handle 110 may be formed of any suitable material such as plastic, polymer, or metal.

In at least some embodiments, the handle 110 is permanently attached to the sheath 102. In other embodiments, the handle 110 is detachable from the sheath 102. In removable-handle embodiments, any suitable temporary attachment mechanism may be employed. In at least some embodiments, the handle 110 is detachable from the sheath 102 by one or more of a snap-fit or a luer-lock arrangement.

The sheath 102 may be formed from any expandable materials suitable for insertion into a patient including, cutouts in a metal tube, elastic plastic with support rings to prevent collapsing, or the like. In at least some embodiments, the sheath 102 is formed as a mesh that includes multiple filaments arranged into one or more helices. In at least some embodiments, the filaments are arranged into two sets of parallel helices wound in opposite directions about the longitudinal length 140 to form walls of the lumen 108. The filaments may intersect each other in an overlapping pattern at multiple interstices, such as interstice 120. The interstices 120 are configured to permit the two filament sets to move with respect to each other, thereby allowing the sheath 102 to axially shorten (i.e., decrease along the longitudinal length 140 of the sheath 102) and radially expand (i.e., increase in diameter 130) when subjected to a compressive force, and conversely, axially lengthen (i.e., increase in longitudinal length 140) and radially contract (i.e., decrease in diameter 130) when subjected to a tensile force. The braid angle θ (i.e., the angle between the two filaments along the longitudinal length 140 of the sheath 102) may be varied to alter the amount of radial expansion/contraction and axial shortening/lengthening of the sheath 102, as desired. Optionally, one (or both) ends of one (or both) sets of filaments may be constrained to prevent the filaments from fraying or unraveling. In at least some embodiments, the sheath 102 includes an expandable single-piece thin-wall metal tube. A pattern may be cut (e.g., laser cut, or the like) in the side wall to create “virtual filaments.”

In at least some embodiments having mesh formed from filaments, the filaments are arranged into alternate configurations. In at least some such embodiments, the sheath 102 is formed from a single set of helices wound in the same direction. In other such embodiments, the sheath 102 may be formed from two helical sets of filaments wound in a first direction and a third helical set of filaments wound in a second direction. It will be understood that the mesh may be formed from other different winding patterns.

The filaments themselves may have any suitable transverse cross-sectional shape. In at least some embodiments, the filaments are formed from multi-filar threads woven together to form filaments having a transverse cross-sectional shape that is, for example, round, oblong, triangular, rectangular, flat, or the like or combinations thereof. In at least some embodiments, the filaments are formed as one or more wires, flat ribbons, threads, fibers, monofilaments, multi-filaments, or the like or combinations thereof.

The filaments may have any suitable transverse cross-sectional thickness. In at least some embodiments, the filament thickness is selected based on a desired amount of resistance to radial expansion of the sheath 102. In at least some instances, the larger the diameter of the filament, the greater the resistance to radial expansion.

The amount of resistance to radial expansion of the sheath 102 may also be, at least in part, attributable to the material composition of the filaments. Any material suitable for inserting into a patient may be used to form the filaments including, for example, polymers, metals, metal alloys, metal-polymer composites, metal-metal composites, or the like or combinations thereof.

Elastomeric materials, such as plastics may also be employed to impart a desired amount of flexibility to the sheath 102. Examples of suitable polymeric materials include, for example, polyethylene terephthalate (“PET”), polytetrafluoroethylene (“PTFE”), polyurethane, fluorinated ethylene propylene (“FEP”) polymers, ethylene tetrafluoroethylene (“ETFE”) polymers, polyurethane, polypropylene (“PP”), polyvinylchloride (“PVC”), polyether-ester, polyester, polyamide, elastomeric polyamides, block polyamide/ethers, polyether block amide (“PEBA”), silicones, polyethylene (“PE”), polyether-ether ketone (“PEEK”), polyimide (“PI”), polyetherimide (“PEI”), polyphenylene sulfide (“PPS”), polyphenylene oxide (“PPO”), polysulfone, perfluoro(propyl vinyl ether) polymers, perfluoroalkoxy (“PFA”) polymers, or the like or other mixtures, combinations or copolymers thereof. Examples of suitable metals or metal alloys may include, for example, stainless steel, platinum, tungsten alloy, nickel-titanium alloy, or the like or combinations thereof.

The sheath 102 may be formed from one or more composite materials configured to impart a desired characteristic to the sheath 102. For example, the sheath 102 can be formed using one or more stainless steel and nickel-titanium alloy wires that are wound together to form filaments having a desired characteristic, such as super elasticity. In at least some embodiments, a composite material may be formed by a drawing, cladding or other suitable process to form the sheath 102 with a desired characteristic.

In at least some embodiments, at least some portions of the sheath 102 may include one or more radiopaque materials for producing a visible image on a fluoroscopic monitor or other imaging device. Suitable materials include, for example, gold, palladium, platinum, tantalum, tungsten alloy, one or more polymeric materials loaded with radiopaque agents (e.g., barium sulfate (BaSO₄), bismuth subcarbonate ((BiO)₂CO₃), or the like), or the like or combinations thereof.

In at least some embodiments, a biocompatible polymer material, such as silicone, may be disposed over the sheath 102, or one or more portions thereof. Such an applied material may be advantageous to reduce, or even prevent, one or more leads inserted into the lumen 108 from undesirably extending through gaps between adjacent filaments along the longitudinal length of the sheath 102.

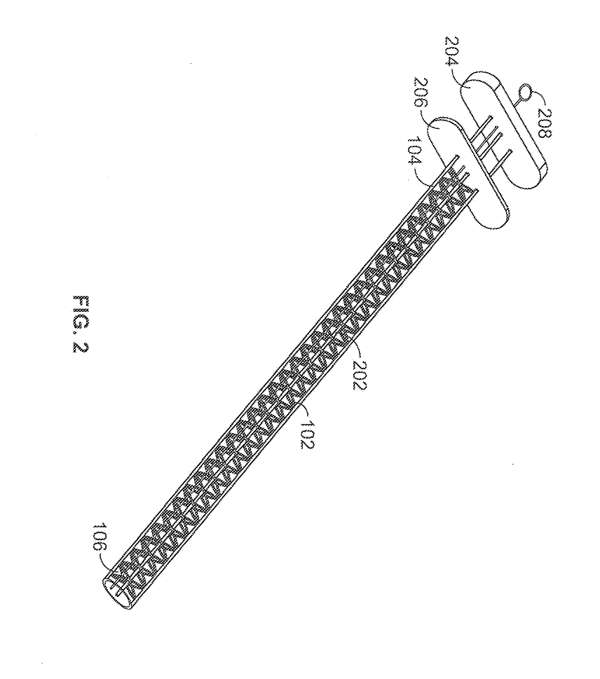

Turning to FIG. 2, in at least some embodiments the insertion assembly includes a securing mechanism for securing the sheath to the insertion needle. In at least some embodiments, the insertion assembly includes a steering mechanism for facilitating guidance of the sheath within the patient, for example, after the sheath has been separated from the insertion needle.

FIG. 2 illustrates an embodiment of the sheath 102 having a steering mechanism for facilitating maneuvering of the sheath 102. In FIG. 2, the steering mechanism includes one or more steering cables 202, a first handle 204, and a second handle 206. The first handle 204 and the second handle 206 are disposed at the proximal end 104 of the sheath 102. The second handle 206 is disposed distally from the first handle 204. The one or more steering cables 202 have a first end coupled to the distal end 106 of the sheath 102, and an opposing second end coupled to the first handle 204 and the second handle 206.

When the insertion assembly is advanced to the epidural space, the sheath 102 may be steered to a target stimulation location for implanting the one or more leads. In at least some embodiments, the distal end 106 of the sheath 102 may be steered by movement of one or more of the steering cables 202.

In at least some embodiments, the steering cables 202 may be used to steer the distal end of the sheath by moving (e.g., rotating, tilting, or both) the first handle 204 and the second handle 206 relative to one another. For example, an operator may hold the first handle 204 stationary and rotate the second handle 206. The rotation deflects the steering cables 202, which, in turn, steers the distal end 106 of the sheath 102 in a direction corresponding to the deflection. In at least some embodiments, the degree and direction of steering may correspond to the degree and direction of handle 110 rotation.

In the illustrated embodiment, the insertion assembly 100 includes four steering cables equidistant from each other around a circumference of the sheath 102. It will be appreciated, however, that the number of steering cables 202 and their placement may vary greatly depending on the degree of steerability desired. It will be understood that any other steering mechanisms (e.g., electrical, mechanical, or both) may be used in lieu of, or in addition to, using one or more steering cables 202.

In at least some embodiments, the insertion assembly 100 includes a mechanism for securing the sheath 102 to the insertion needle (150 in FIG. 10). FIG. 2 shows the insertion assembly 100 including a pull string 208 for securing the sheath 102 to the insertion needle (150 in FIG. 10). It will be understood that any other suitable sheath-securing features (e.g., one or more clamps, pull-away members, or the like or combinations thereof) may be used in lieu of, or in addition to, using one or more pull strings 208.

The pull string 208 extends from the distal end 106 of the sheath 102 to a location in proximity to the first handle 204. In at least some embodiments, the pull string 208 is used to secure the sheath 102 around at least a portion of the insertion needle (150 in FIG. 10). Securing the sheath 102 to the insertion needle may prevent the sheath 102 from separating from the insertion needle during insertion of the insertion assembly into the patient. The pull string 208 may also prevent the sheath 102 from undesirably expanding radially, for example, prior to disposing at least a portion of the sheath 102 in the epidural space.

The pull string 208 may be used to facilitate radial expansion of the sheath 102. In at least some embodiments, actuating the pull string 208 (e.g., pulling the pull string 208) may cause the sheath 102 to expand radially. For example, the pull string 208 may couple to one or bands, or the like that fit over a portion of the sheath 102 and that prevent the sheath 102 from expanding. In which case, actuating the pull string 208 causes the bands to move, or break, or the like to enable the sheath 102 to expand.

In at least some other embodiments, actuating the pull string 208 causes the sheath 102 to tighten against the insertion needle. For example, the pull string 208 may cinch the distal end 106 of the sheath 208, thereby tightening the sheath 102 against the insertion needle 150 when the pull string 208 is actuated. In at least some embodiments, actuating the pull string 208 causes the sheath 102 to tighten against the one or more leads. For example, when the insertion needle is removed from the sheath and the one or more leads are inserted into the sheath, the pull string 208 may be used to cinch the distal end 106 of the sheath 102 against the one or more leads disposed therein, thereby tightening the sheath 102 against the one or more leads.

In at least some embodiments, the unsecured end of the pull string 208 is positioned such that it is accessible to a medical practitioner when the distal end 106 of the sheath 102 is inserted into the patient. In at least some embodiments, the pull string 208 may be actuated by pulling the pull string 208 proximally (i.e., in a direction opposite to the distal end 106 of the sheath 102).

In at least some embodiments, this motion places a tensile force on the sheath 102, extending it longitudinally while compressing it radially so that it fits snuggly around the insertion needle. A snug fit between the sheath 102 and the insertion needle (150 in FIG. 10) may enable the insertion assembly to more easily enter small cavities in a patient's body.

In at least some other embodiments, once the pull string 208 is actuated, the tensile force on the sheath 102 is removed. Consequently, the sheath 102 may expand radially, thereby radially expanding the passage formed by the insertion assembly. The insertion needle may then be removed and one or more leads advanced through the lumen of the sheath and into the epidural space.

In at least some embodiments, the sheath 102 may be reusable. Using one or more pull strings (or other suitable mechanism, such as one or more levers) to place tensile force on the sheath 102 during a lead implantation procedure may enable operators to reuse the insertion assembly 100. For example, if an operator inadvertently radially expands the sheath 102 in a wrong position and removes the insertion needle, the operator may desire to retrieve the sheath 102 for replacement. But, because the sheath 102 is radially expandable, it may get wedged between muscles, ligaments, tissues, or any other such impediments, which may increase difficulty in extracting the sheath 102. In such situations, the operator may reinsert the insertion needle 150 into the lumen 108 of the sheath 102 and pull on the pull string 208 to radially compress the sheath 102 to re-fit the sheath 102 around the insertion needle 150. The insertion assembly 100 may then be removed from the patient or replaced in the patient.

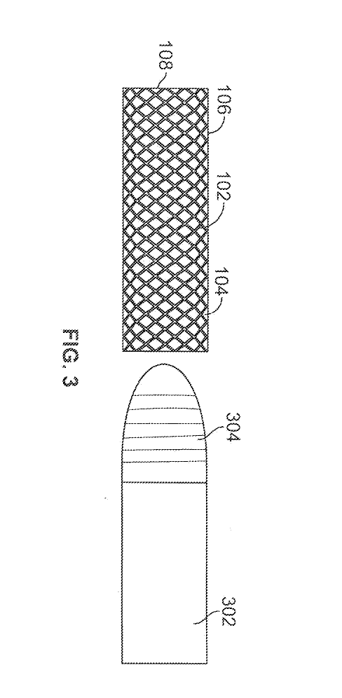

Turning to FIG. 3, in some cases once a sheath is expanded in a patient, it may be desirable to insert a tissue-separating member into the sheath 102 to separate tissue which may prevent one or more portions of the sheath 102 from fully expanding. In embodiments of the sheath where the sheath is not compressed against the insertion needle, the tissue-separating member may used to apply a force to radially expand the sheath.

FIG. 3 is a schematic side view of another embodiment of a portion of the sheath 102 and a portion of a tissue-separating member 302. In FIG. 3, the tissue-separating member is shown as a flexible passing elevator 302. In at least some embodiments, the tissue-separating member 302 is configured and arranged for coupling to one or more leads and transporting the one or more leads along the lumen 108 of the sheath 102.

The passing elevator 302 may be narrower or wider than the sheath's lumen 108. In the latter case, the sheath 102 may expand to accommodate the elevator 302. In at least some embodiments, the one or more leads may be transported to the distal end 106 of the sheath 102 by placing them on the passing elevator 302 and pushing the passing elevator 302 along the sheath 102. In embodiments using the passing elevator 302, the insertion assembly 100 may not include the one or more steering cables 202 because, once the insertion needle is removed, the steering function may performed by the passing elevator 302. In at least some embodiments, the passing elevator 302 has a distal portion 304 that is more flexible than remaining portions of the passing elevator 302. Forming the passing elevator 302 with a more flexible distal end 304 may improve the ability of the passing elevator 302 to circumvent obstructions without damaging any body organs, muscles, tissues, or bones in the epidural space.

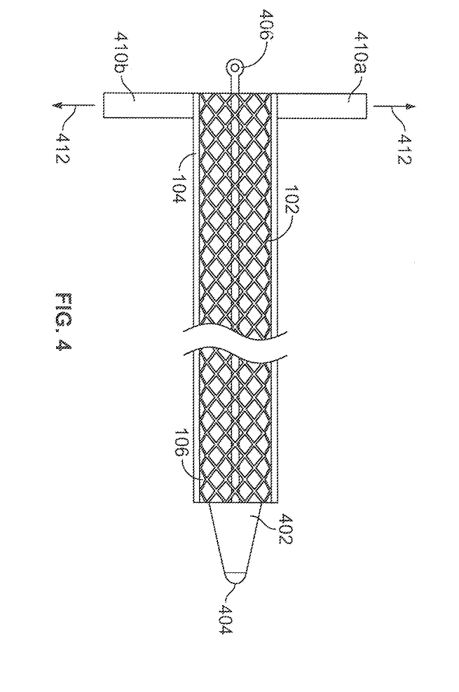

In at least some embodiments, it may be desirable to use a stiffening member for assisting the advancement of the tissue-separating member through the sheath. FIG. 5 is a schematic side view of one embodiment of the sheath 102 with a stiffening member 504 disposed in the sheath 102 and abutting the tissue-separating member 302. In FIG. 5, the tissue-separating member 302 is shown guided through the sheath 102 such that the tissue-separating member 302 extends distally beyond the distal end 106 of the sheath 102. In at least some embodiments, the tissue-separating member 302 is pushed through the sheath 203 using the stiffening member 504.

The stiffening member 504 provides enough rigidity to push the tissue-separating member 302 through the sheath 102, while the tissue-separating member 302 is more flexible than the stiffening member 504 and is capable of bending and steering past obstacles without damaging organs. Once the tissue-separating member 302 is extended through the sheath 102, the stiffening member 504 may be retracted and the one or more leads may be inserted. In at least some embodiments, the one or more leads may be introduced in the sheath 102 while the stiffening member 504 remains within the sheath 102.

Turning to FIG. 4, in some cases when the insertion needle and sheath are advanced to the epidural space, the insertion needle removed, and the one or more leads disposed in the sheath, it may be desirable to use a guiding member to assist with guiding the sheath and lead(s) to the target stimulation location. In some cases, it may be desirable to include multiple tabs that are each coupled to the proximal end of the sheath and that can be pulled outwardly to separate from one another, thereby stretching the sheath radially.

Turning to FIG. 4, in at least some embodiments after the insertion needle is removed a guiding member may be advanced from a distal end of the sheath to facilitate guidance of the sheath and one or more leads to the target stimulation location. FIG. 4 is a schematic side view of another embodiment of the sheath 102 with a guiding member 402 disposed at the distal end 106 of the sheath 102. Pull-apart tabs 410a and 410b are coupled to the proximal end 104 of the sheath 102. The pull-apart tabs 410a and 410b are configured for enabling a medical practitioner to pull the pull-apart tabs 410a and 410b away from one another along the directions shown by arrows 412. In at least some embodiments, pulling apart the pull-apart tabs 410a and 410b along the directions of the arrows 412 causes the proximal end 104 of the sheath 102 to stretch, thereby increasing the diameter of at least a portion of the sheath 102.

The pull apart tabs 410a and 410b enable the proximal end 104 of the sheath 102 to expand, providing greater space for receiving one or more leads, such as leads with larger-diameter sections (e.g., a larger-sized junction, or the like), or other irregularly shaped object. For example, some splitters (such as the 2×4 splitter by Boston)Scientific° have junction diameters larger than lead diameters.

The guiding member 402 includes a distal end 404 and a proximal end 406. The proximal end 406 of the guiding member 402 may be accessible to a medical practitioner and used to control advancement of the guiding member 402 along the sheath 102. In at least some embodiments, the proximal end 406 of the guiding member 402 extends beyond the proximal end 104 of the sheath 102.

In at least some embodiments, the distal end 404 has a diameter that is smaller than the diameter (130 in FIG. 1) of the sheath 102 at the distal end 106. In at least some embodiments, the distal end 404 is flexible enough to steer the distal end 106 of the sheath 102 to a desired location and to provide angular displacement for placing the one or more leads once the distal end 106 of the sheath 102 is in the epidural space and the one or more leads are disposed in the sheath 102.

In at least some embodiments, the guiding member 402 is retractable. In which case, the guiding member 402 may be retracted into the sheath 102 during advancement of the insertion assembly into the patient, and transitioned to an actuated state (where the distal end of the guiding member 402 extends beyond the distal end 106 of the sheath) when needed for guidance. Any suitable actuation mechanism may be employed to actuate the guiding member 402.

In at least some embodiments, pushing the proximal end 406 of the guiding member 402 towards the distal end 404 of the guiding member 402 actuates the guiding member 402, and pulling the proximal end 406 of the guiding member 402 proximally retracts the distal end 404 of the guiding member 402 into the distal end 106 of the sheath 102. It will be understood that many other such actuation mechanisms may be contemplated without departing from the scope of the present disclosure.

During an exemplary procedure, the guiding member 402 may be retracted when the sheath 102 is inserted into the patient. When the sheath 102 is placed, the one or more leads may be inserted into the sheath 102. The guiding member 402 may be actuated and the flexible distal portion 404 of the guiding member 402 may then be steered to provide appropriate trajectory for the one or more leads to be placed in the epidural space.

In at least some embodiments, the insertion assembly 100 includes a steering mechanism (e.g., a mechanical, electronic, or electromechanical mechanism) to steer the distal end 404 of the guiding member 402. In at least some embodiments, orientation of the guiding member 402 is controllable by rotation of the sheath 102. The guiding member 402 may be formed of a suitable biocompatible metal, plastic, or polymer material. Moreover, the guiding element may include radiopaque materials (e.g., markers, bands, or the like) to enable physicians to monitor the position, or orientation, or both of the guiding member 402 with respect to the epidural space. In at least some embodiments, the guiding member 402 includes one or more support regions, or substantially flat surfaces, or both at the distal end 404 for supporting the one or more leads before they are placed in the epidural space.

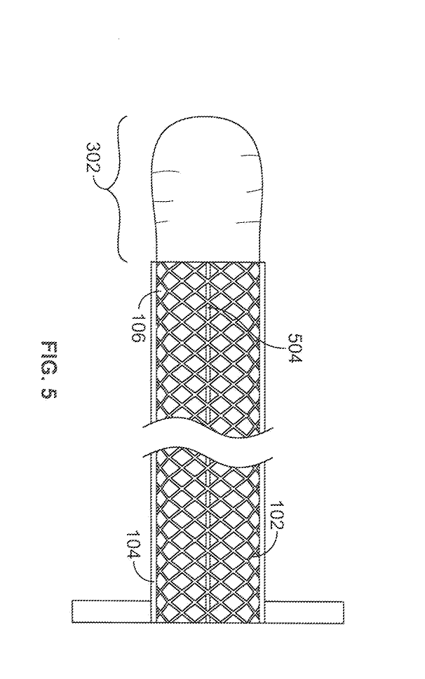

Turning to FIGS. 6A-6B, in some instances it may be desirable to radially expand only a portion of the sheath at a time. For example, when implanting a lead with an over-sized junction at the proximal end of the lead, the distal end of the lead may be advanced along the sheath without radially expanding the sheath. Advancement of the proximal end of the lead, however, may require radial expansion of the sheath. In which case, the over-sized junction at the proximal end of the lead may be accommodated by radially expanding the proximal end of the sheath so that the sheath is only expanded in the region disposed over the over-sized junction. This may be performed in any suitable way including, for example, pulling apart pull-apart tabs coupled to the proximal end of the sheath (see e.g., FIG. 4).

In some instances, however, once the lead with the over-sized junction at the proximal end of the lead is placed, retraction of the sheath may cause the lead to move out of position. For example, in some cases the junction may become entangled in the sheath 102 during retraction and inadvertently move distally, thereby displacing the lead. In other cases, the sheath 102 may fit tightly around the junction and, when being retracted, pull the junction along with it.

In at least some embodiments, the insertion assembly includes a local sheath expansion system that enables a radially expanded region of the sheath to be movable along the longitudinal length of the sheath. Thus, it may be advantageous for a locally expanded region of the sheath to be able to move along the longitudinal length of the sheath in order to remain disposed over the over-sized junction during removal of the sheath from the lead, thereby preventing entanglement with the over-sized junction and potentially causing the lead to move after the lead has been placed.

FIG. 6A is a schematic perspective view of an embodiment of the sheath 102 with a local sheath expansion system 650 for radially expanding one or more selected portions of the sheath. In FIG. 6A, the sheath 102 is shown disposed in a first position, where the sheath 102 is isodiametric and not expanded. FIG. 6B is a schematic perspective view of a portion of the sheath 102 in a second position, where the sheath 102 includes a radially expanded portion, and a lead 612 with an oversized junction 614 partially disposed in the sheath 602.

The local sheath expansion system 650 includes a rail 602 and a sheath expanding member 604. The rail 602 extends longitudinally along the outer surface of the sheath 102, from the proximal end 104 to the distal end 106. The sheath expanding member 604 engages the rail 602 such that the sheath expanding member 604 is translatable along a longitudinal length of the sheath 102, via the rail 602. In at least some embodiments, an inner surface of the sheath expandable member 604, an outer surface of the rail 602, or both, may be coated with a lubricious material to assist in translation of the sheath expandable member 604 along the rail 602. Optionally, a proximal end, a distal end, or both, of the rail 602 may include a coupling member (e.g., one or more elastic rings, magnetic locks, projections, upward curving surfaces, or the like) 606 for coupling the rail 602 to the sheath 102.

The sheath expanding member 604 includes an expandable flange 608 and a locking assembly 610. The expandable flange 608 is coupled to the rail 602 and to the sheath 102. The expandable flange 608 laterally surrounds at least a portion of the circumference of the sheath 102 and can be expanded. Expansion of the expandable flange 608 causes a corresponding expansion of the sheath 102 at the location of the expandable flange 608.

In at least some embodiments, the locking assembly 610 is configured for locking the expandable flange 608 at a desired level of radial expansion such that the portion of the sheath 102 coupled to the expandable flange 608 has a diameter suitable for receiving the junction 614 of the lead 612. The locking assembly 610 includes an adjustable-length fastening assembly, such as an expansion latch 620 configured and arranged to mate with a retaining feature 622 (e.g., a post, pin, or the like). In at least some embodiments, the expansion latch 620 is disposed on a first end of the expandable flange 608 and the retaining feature 622 is attached to a second end of the expandable flange 608, opposite to the first end. As shown in FIG. 6B, the expansion latch 612 is configured and arranged to mate with the retaining feature 614 at any one of multiple different locations, each mating location locking the sheath 102 at a different level of radial expansion.

It will be understood that any suitable sheath expanding member 604 that enables a circumference of a region of the sheath 102 to expand radially, and maintain that level of radial expansion, may be utilized without departing from the scope of the present disclosure. For example, in at least some embodiments the sheath expanding member 604 includes a substantially circular ring with an adjustable diameter.

In one example of an expansion operation, the insertion assembly 100 is introduced into the patient. The insertion needle is removed and the lead 612 is introduced into the sheath 102. The sheath expanding member 604 is placed at the proximal end of the sheath 102 and expanded and locked at a level of radial expansion suitable for receiving the junction 614. The lead 612 is advanced in the sheath until the junction 614 is disposed under sheath expanding member 604. If further advancement of the lead 612 is desired, the sheath expanding member 604 may be moved with the junction 612 along the longitudinal length of the sheath 102. When the lead 612 is placed, the sheath 102 may be removed from the patient. As the sheath 102 is refracted proximally, the sheath expanding member 604 can be moved distally along the length of the sheath 102 such that the sheath expanding member 604 remains disposed over the junction 614. By maintaining position of the sheath expanding member 604 over the junction 614 while the sheath 102 is retracted proximally, the sheath expanding member 604 maintains an expanded region around the junction 614 to accommodate the junction 614 along the length of the sheath 102, thereby preventing the junction 614 from being undesirably pulled by the sheath 102 during retraction and, potentially, displacing the distal end of the lead 612 from the target stimulation location.

Turning to FIG. 7, in at least some embodiments the sheath has a body that tapers from the proximal end to the distal end. It may be advantageous to design the sheath to taper distally to facilitate removal of underlying components (e.g., the insertion needle, the tissue-separating member, or the like or combinations thereof) during a lead implantation procedure. FIG. 7 is a schematic side view of one embodiment of a sheath 102 having a distally-tapering body. In FIG. 7, the proximal end 104 of the sheath 102 has a first diameter 704 and the distal end 106 of the sheath 102 has a second diameter 706 that is smaller than the first diameter 704.

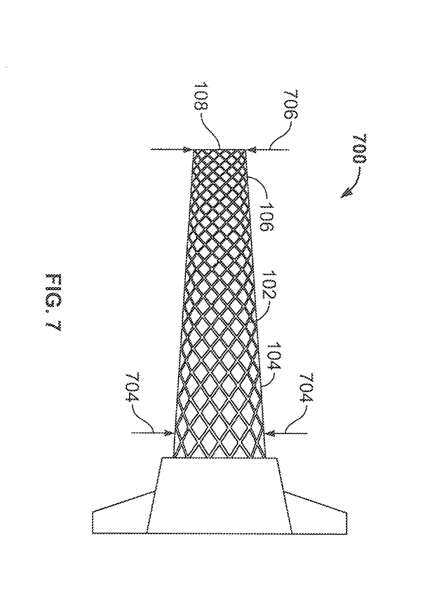

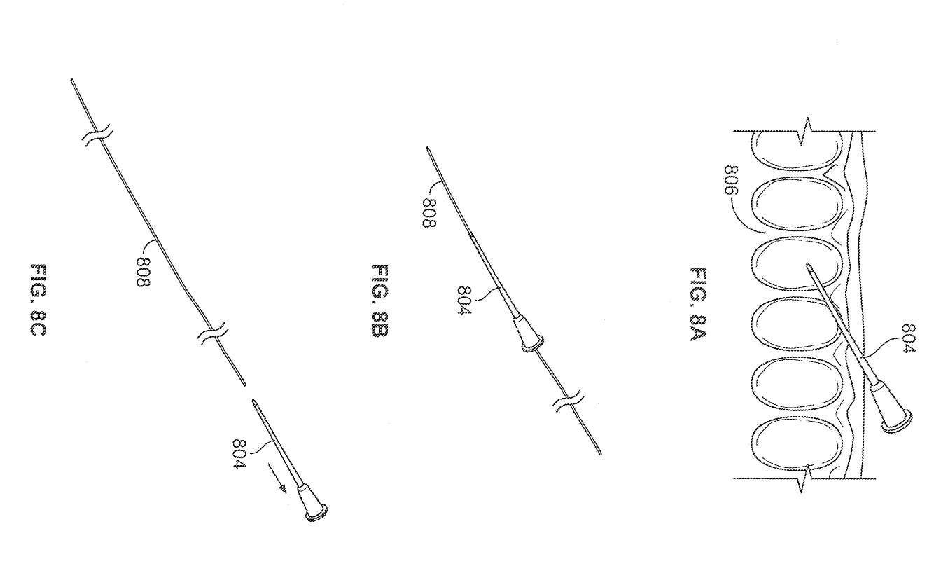

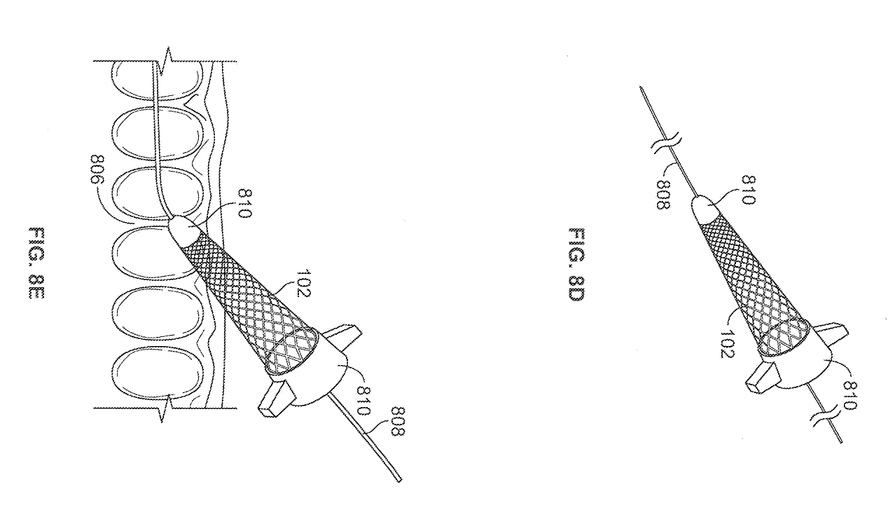

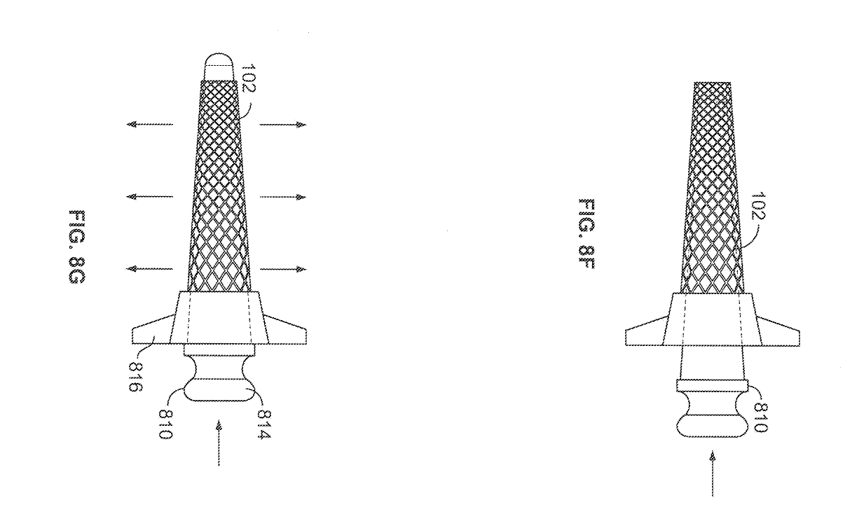

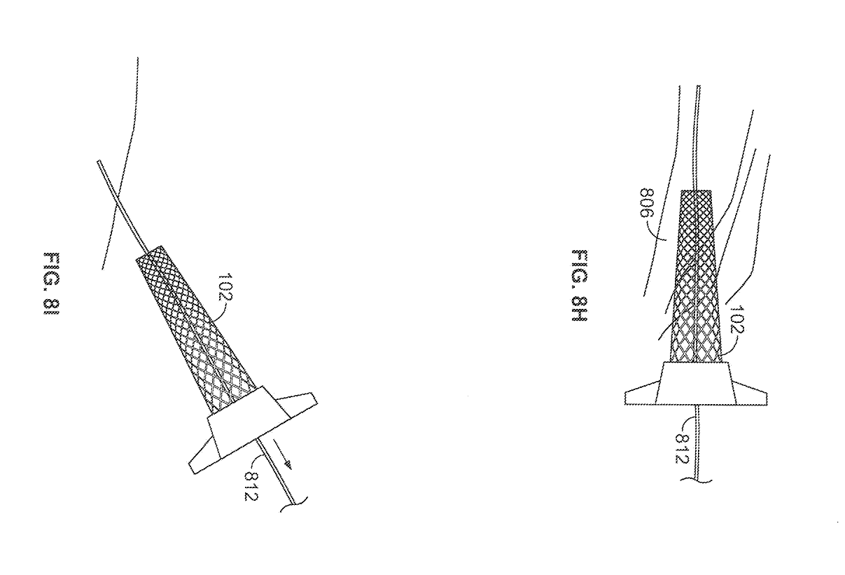

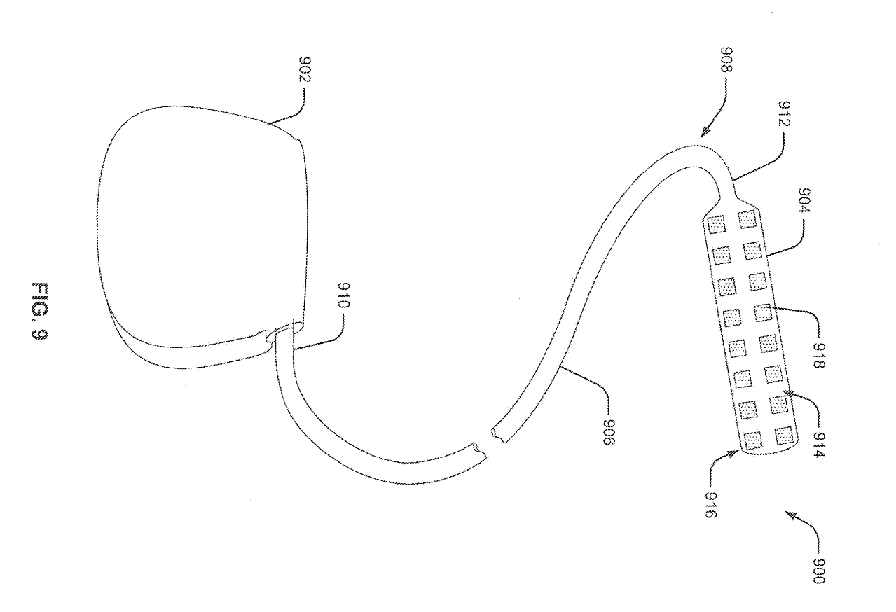

Turning to FIGS. 8A-8I, in at least some embodiments the insertion assembly includes a guidewire for facilitating delivery of the sheath. FIGS. 8A-8I collectively illustrate one narrow embodiment of implanting a lead using an assertion assembly that includes a guidewire.

FIG. 8A shows one embodiment of an insertion needle 804 inserted into the epidural space 806 of a patient. Optionally, confirmation may be achieved that the distal end of the insertion needle 804 is disposed in the epidural space 806 (e.g., a loss of resistance test, or the like may be performed). FIG. 8B shows a guidewire 808 inserted distally through a lumen of the insertion needle 804 until a distal end of the guidewire extends through a distal end of the insertion needle 804. When the distal end of the guidewire 808 is in place, the insertion needle 804 may be removed. FIG. 8C shows the insertion needle 804 being removed from a proximal end of the guidewire 808, while leaving the distal end of the guidewire 808 in place.

FIG. 8D is a schematic perspective view of one embodiment of the sheath 102 disposed over a tissue-separating member 810. In FIG. 8D, and in other figures, the sheath 102 is shown as being distally tapering (see e.g., FIG. 7). In FIG. 8D, and in other figures, the tissue-separating member 810 is shown as a dilator. The distal end of the dilator 810 defines an opening that extends along a longitudinal length of the dilator 810 and that is configured and arranged to receive the guidewire 808. In at least some embodiments, after the insertion needle 804 is removed from the guidewire 808 the sheath 102 may be disposed over the dilator 812 and the sheath 102 and dilator 812 may be guided along the guidewire 808. FIG. 8E illustrates the sheath 102 and the dilator 812 guided over the guidewire 808 and inserted into the epidural space 806.

FIG. 8F illustrates one embodiment of the sheath 102 partially inserted into the dilator 810. FIG. 8G illustrates one embodiment of the sheath 102 fully inserted into the dilator 810. In FIGS. 8F-8G and in other figures, the sheath 102 and the dilator 810 are shown as being distally-tapering. It may be advantageous for the sheath 102 and the dilator 810 to both taper distally to facilitate separation and removal of the dilator 810 from the sheath 102 after the sheath 102 is positioned in the patient. Tapering of the sheath 102 may also provide a mechanical technique for expanding the sheath 102 within patient tissue, thereby providing an enlarged passage to accommodate the one or more leads. For example, pushing the dilator 810 into the sheath 102 may cause the sheath 102 to expand, as shown in FIGS. 8F-8G.

In some embodiments, the dilator 810 is shaped similar to the sheath 102 but with a different taper angle. Preferably, the dilator 810 may have a greater taper angle than the sheath 102. In at least some embodiments, the dilator 810 has a diameter that is larger than the diameter of the sheath 102. Optionally, the proximal end of the dilator 810 may include a plunger cap 814 and the proximal end of the sheath 102 may include a flanged handle 816. In which case, a medical practitioner may expand the sheath 102 by squeezing his or her fingers between the plunger cap 814 and flanged handle 816, in a manner similar to using a syringe.

In at least some embodiments, the sheath 102 and the dilator 810 have transverse cross-sections that are shaped to receive the paddle body (904 in FIG. 9) (e.g., have a major axis and a minor axis that is perpendicular to the major axis and that is smaller than the major axis). In at least some embodiments, the dilator 810 has a proximal end that is formed from one or more materials having a lower durometer than the distal end to potentially reduce the risk of trauma to patient tissue during an implantation procedure. In at least some embodiments, the dilator 810 is isodiametric. In at least some embodiments, the sheath 102 is isodiametric. It will be understood that any suitable tissue-separating member may be used in lieu of, or in addition to, using the dilator 810 including, for example, one or more balloons inflatable with one or more gasses (e.g., air, carbon dioxide, nitrogen, or the like or combinations thereof).

In at least some embodiments, the sheath 102 is advanced over the guidewire 808 with the dilator 810 partially disposed inside the lumen 108 of the sheath 102. Once the sheath 102 is disposed in proximity to the target stimulation location, the dilator 810 may be pushed distally within the sheath's lumen 108, thereby expanding the sheath 102. In at least some embodiments, the extent of expansion may be determined, at least in part, by the size of the dilator 810. Increasing at least one of the taper angle difference or the diameter difference between the sheath 102 and the dilator 810 may have a corresponding increase in the amount of expansion of the sheath 102.

In at least some embodiments, when the sheath 102 is placed and expanded the dilator 810 may be removed from the patient. One or more leads may be transported along the lumen 108 of the expanded sheath 102. FIG. 8H illustrates one embodiment of a lead 812 extending through the sheath 102. Once the one or more leads 812 are inserted and placed, the sheath 102 may be compressed and retracted, or refracted without compression, as illustrated in FIG. 8I. In at least some embodiments, the sheath 102 may remain implanted in the patient at the end of the implantation procedure, in either an expanded or contracted position.

Embodiments of the present disclosure may be used in any medical or non-medical procedure, including any medical procedure where one or more body part requires electrical stimulation. In addition, at least certain aspects of the aforementioned embodiments may be combined with other aspects of the embodiments, or removed, without departing from the scope of the preset disclosure.

While the present disclosure has been described by means of specific embodiments and applications thereof, numerous modifications and variations could be made thereto by those skilled in the art without departing from the scope of the present disclosure set forth in the claims.

What is claimed is:

1. An insertion assembly for assisting implantation of at least one lead into a patient, the insertion member comprising:
an insertion needle configured and arranged for advancing into the patient, the insertion needle having an outer surface; and
a sheath having a proximal end with a first diameter, a distal end with a second diameter, and a lumen extending between the proximal and distal ends, the sheath radially expandable from a non-expanded state to an expanded state that is rigid enough to retract surrounding patient tissue when the sheath is inserted into the patient during implantation, the sheath configured and arranged for removably coupling with the insertion needle while the insertion needle is being advanced into the patient, wherein when the sheath is coupled to the insertion needle the sheath is disposed over at least a portion of the outer surface of the insertion needle, and wherein when the sheath is in an expanded state the first and second diameters are each large enough to concurrently receive at least one of a paddle lead or at least two percutaneous leads.

2. The insertion assembly of claim 1, further comprising a tissue-separating member insertable into the lumen of the sheath, the tissue-separating member configured and arranged for facilitating the transition of the sheath to an expanded state.

3. The insertion assembly of claim 2, wherein the tissue-separating member comprises at least one of a passing elevator or a dilator.

4. The insertion assembly of claim 1, further comprising at least two pull-apart tabs disposed at the proximal end of the sheath.

5. The insertion assembly of claim 1, further comprising a first handle disposed at the proximal end of the sheath.

6. The insertion assembly of claim 5, further comprising a second handle disposed at the proximal end of the sheath, and wherein the second handle axially spaced-apart from the first handle along the longitudinal length of the sheath.

7. The insertion assembly of claim 6, further comprising at least one steering cable that is coupled to the distal end of the sheath and extends to at least one of the first handle or the second handle.

8. The insertion assembly of claim 1, further comprising a retractable guiding member disposed at the distal end of the sheath, the guiding member for assisting with guiding the sheath through patient tissue.

9. The insertion assembly of claim 1, further comprising at least one pull string coupled to the distal end of the sheath and extending in proximity to the proximal end of the sheath.

10. The insertion assembly of claim 1, further comprising at least one stiffening member configured and arranged for insertion into the lumen of the sheath, the at least one stiffening member for assisting with guiding the sheath through patient tissue.

11. The insertion assembly of claim 1, wherein the first diameter is equal to the second diameter.

12. The insertion assembly of claim 1, wherein the first diameter is larger than the second diameter.

13. The insertion assembly of claim 1, further comprising a local sheath expansion system configured and arranged for radially expanding a cross-sectional portion of the sheath while not radially expanding at least one other cross-sectional portion of the sheath, the local sheath expansion system comprising
a rail extending along the longitudinal length of the sheath; and
a sheath expanding member coupleable to the rail, the sheath expanding member configured and arranged to radially expand a cross-sectional portion of the sheath and retain the expanded cross-sectional portion of the sheath in the expanded state, wherein the sheath expanding member is translatable along the rail.

14. An insertion kit comprising
the insertion assembly of claim 1; and
a first lead having a proximal end and a distal end, the first lead comprising
a lead body having a first diameter,
a plurality of electrodes disposed at the distal end of the lead body,
a plurality of terminals disposed at the proximal end of the lead body, and
a plurality of conductors electrically coupling at least one of the electrodes to at least one of the terminals;
wherein the lead body is configured and arranged for insertion into the lumen of the sheath when the sheath is disposed in an expanded state.

15. The insertion kit of claim 14, wherein the first lead comprises a junction disposed along the lead body, and wherein the junction has a diameter that is larger than the first diameter of the lead body.

16. The insertion kit of claim 14, wherein the first lead is a paddle lead.

17. An electrical stimulation system comprising
the insertion kit of claim 14; and
a control module coupleable to the proximal end of the first lead.

18. The insertion kit of claim 14, further comprising at least one second lead having a proximal end and a distal end, the at least one second lead comprising
a lead body having a first diameter;
a plurality of electrodes disposed at the distal end of the at least one lead body;
a plurality of terminals disposed at the proximal end of the at least one lead body; and
a plurality of conductors electrically coupling at least one of the electrodes to at least one of the terminals;
wherein the lead body of the at least one second lead is configured and arranged for insertion into the lumen of the sheath concurrently with the lead body of the at least one first lead when the sheath is disposed in an expanded state.

19. A method of inserting a lead into a patient, the method comprising
advancing the insertion assembly of claim 1 into the patient;
uncoupling the sheath from the insertion needle;
transitioning the sheath to an expanded state;
removing the insertion needle from the patient;
inserting a first lead into the lumen of the sheath when the sheath is in an expanded state;
inserting at least one second lead into the lumen of the sheath when the sheath is in an expanded state, wherein the first lead and the at least one second lead are concurrently disposed in the sheath;
advancing the first lead to a first target stimulation location in the patient along the lumen of the sheath; and
advancing the at least one second lead to a second target stimulation location in the patient along the lumen of the sheath.

20. An insertion assembly for assisting implantation of at least one lead into a patient, the insertion assembly comprising:
a guidewire configured and arranged for insertion into the patient;
an insertion needle configured and arranged for advancing into a patient via the guidewire, the insertion needle having a proximal end and a distal end, the insertion needle defining a lumen extending between the proximal and distal ends, the lumen configured and arranged to receive the guidewire;
a tissue-separating member having a proximal end, a distal end, and an outer surface, the tissue-separating member defining a lumen extending between the proximal and distal ends, the lumen configured and arranged for receiving the guidewire; and
a sheath having a proximal end with a first diameter, a distal end with a second diameter, and a longitudinal length, the sheath defining a lumen extending between the proximal and distal ends, the sheath radially expandable from a non-expanded state to an expanded state that is rigid enough to retract surrounding patient tissue when the sheath is inserted into the patient during implantation, the sheath configured and arranged for removably coupling with the tissue-separating member while the tissue-separating member is being advanced into the patient, wherein when the sheath is coupled to the tissue-separating member the sheath is disposed over at least a portion of the outer surface of the tissue-separating member, and wherein when the sheath is in an expanded state the first and second diameters are each large enough to concurrently receive at least one of a paddle lead or at least two percutaneous leads.

21. The insertion assembly of claim 20, wherein the first diameter of the sheath is larger than the second diameter of the sheath.

22. The insertion assembly of claim 20, wherein the lumen of the sheath has a non-circular shape along an axis transverse to the longitudinal length of the sheath.
